SEQUENCE LISTING (6 sequences)

SEQ ID NO: 1 - DNA, 7495 bp, Ljungan virus
  M1146 strain
agaatagccaatctgtaacttcaacacctaagcgcatcttgcccgccgttatctggtcgaaggcacttgcaataagactggttaaccctccatgcgtgattcacaatcctatctgtctctcgagtgctttacgctcgggtaggggctcacccaggaggtcccactctttacagaggtctgcgcaggtggctttcacctctcgacagcgcccctcaatcctgccttaggtgtgaaggttgcacgtattccaatgtgaacagtgcttgaattttgttggtaggtgtagcgaccacacatgagccagcggatttcccctggtggtaacaccagcctctgggcccaaaaggcatgtctttgacctcatgtgtgcacaatcccagtgatgccaatgattagtaaagcacttagtatatatatgggatataccaatctcaagcacctaagggtgtaggtatggtaggctgaaggatgccctggaggtacccgcaggtaacgattggttcactgtggatctgaccaggggcccaccggaacttcgttctgggtagaagctctcgagccttgggttaaaaaacgtctaggcctatcccccggggggacccggggtttcccttataaccccatattttgcatcatggctggccgtaacgaaactaacgtttctgagcttctctccactgtctcatcaactgtcggatctctgctccagaaccccactgttgaagagaaagaaatggattccgatagggtagctgcgtcaaccacaacaaatgctggcaacttggttcaggcatcagttgctcctactatgccatttaagccagattttaccaatgttgatcagtttttatgtatgagctatagccctgagactgcccctactaacccaaccaaaatggtgcatttggctgctgcacaatggcaaaccacacataatcgcactcatgaagtcctacggattgaattgccacggtcattctggccaaatgaaaattatcctgcatggggtcagtcaagatatttcggagctgtccgttgtggattccattttcaggttcaacttaatgttaacattgggtctgctggctgtttgatagcctgctatttgccagcaacagctttggaaaattttaattcctatacttttggcacctttacaaattttccccatgtattaatgaatgcagccacaacctcccaagctgatttgtatattccctatgtttttaatcataattatgccagaacagattcagcagatttgggtgggatttttatttatgtttggtcagccctaacagttcccaccggcgccccaaccactattgatgtgacagtttttggatctcttttggatttggattttcagaatcccagggtgccaggaacagccattgggatattttctcagggtagaagacaaatgcggaagaccaagtcatctaaatttaaatggacccgaaataaagtggatattgctgaggggcctggtgcaatgaatattgcaaatgttttgtccacaactggtgggcaatcaactgctttggttggtgagagagcattctatgatccccgtacagccggcgcctcgatccgctgtaaagacctaatggatttggcacgcatgccatcatgcttgcgtggtgaaagagcagtaccacaagatactacaggctattttacctggtcatcaaatatcacaccattaaatttcatttttgagtatggagtttattttgaggatattcccaatctgaatctgatggctgccaattttaattattggagaggatccattgtgattaagttaactgtttttgcctcaacttttaataaaggccgtcttcgtatggcatttttcccaaaccaaggtgaaaattttggcgaaccagcagcccaaaattcaatttatgtggtttgtgatataggcttaaataatacatttgaactcactataccctatacgtggggcaattggatgagaccgtcacgtggcggccccgtgggatggctgaggatagatgttttgaatagattaacttttaatagctcatcaccttcctctgttaattgcattttgcaagtgaaaatgggcaatgatgctcagtttatggtgcccactacctccaatttggtttgggagtcacctactggaatgtcctcctggggctcacaaatggacttgattgattcattggataacccagaggagatccaagatagtgaggaacctgaatcatcaaatgttgaagcagcacaggggcaggaggcagcaacagcagtgggtttgagagccactgaaaacgatggttctctaactgagcaagtcaacgtggctcagcctatgttcttgaattttaaacagcataaagttgatatttattcaacatcccataccaaggtggatcatatttttgggagggcttggcgctttcaaattaccaatttggataacagcagtattgggcgctttaccattcccttccccaccaccactcatggatcccttgctagattctttgcctattggacaggcgagttaaatatacatgttattaatatttccacaacaaatgcttttttgaaagttgcgcatacctggtttggcaccacatcagggatagcaagaactggtagtttagagtccaatggtgtgatgataattccgccaaatgagcaaatgactttttgtgttccatattattctgaagctccactacggactgttaagggcactggactctcagccggtcttggcacttttttctatcaggcagttgggcgaaccattcagaatcgcatggagatttttgtctccttgcggtgtccaaatttctttttcccagtaccagccccacatgaagctagtgcaagatcaacactggctagaattgatacagcaactgaggaagagctcaatgctattatgagctctgctgagcccgacacacctctccgcattggtaagccacctgaagatcccttaaaacaattacgtgaggcagcaaaaacctattttaaaatttaccatgataaggatatggattatgctggagggaagtttttaaatcaatgtggtgatgttgagaccaaccctgggcccgatattgaattggtttataaaccccgtggcttttataagcattatggggttagaataaatgggcatgtttatcatttaaattcacaagatattttaaccaccgcaataacgggaaaagcagagtttattaaacaacaagatgatggccattgggttcatgccatgtcagccccccttgattattttactgaaaaatatgttaattctctggttggctctaagcatatattttcagcaactgagaattgtgaaaccatcgctagagatatttttccaggtcaaccagaaatttctcagtctaaagccttgggcatcataggcttaattcttttgtctgcagcactgttgtctcttttggctgtgccctgggattttagttccttgcaaattgtttataatcaaggcattgaaggggatgctagtggtttaactcttttgagtcagcgctgcatgacttttttttccaatactatgtgtgaaacctttaataatgatttagtgaaatttataattaaaattttggttaggcttttatgttatattgtgctttattgtcatgctcctaatatgcttacaaccatgtgcctggggactttacttgtgctggatattacaacatgtgagatcttatcagccaacaccaaggcattgtttcaggccctaatggatggagatgtgaagagtttggtctggaaaattgctgaaaatatgcaatttgctcaatcatctgatgaacaagcagaagagatgtctgccactttttcatttgctaaggatatggttgatatacattcacctgatcaaccaccctttcagaatcagagttggagggattttaatgaggttagtatgtcttttagacatgttgagtggtggttaacaatgtttaagaagatttataatgttttgaaaggcattttctcccctagcattgaacaaaaagctgtggcatggcttgaaaggaaccatgagtatgtggcaggtgtcttggaccagtgttctgatatgatcattaagatgaaggatcccaaacagcaaaggaatgctaaggtcattgaggaatattttgaagtcttgaaacgcatgaagcccttggtgtctttgtgtattaaagtggctccctccaccaaattctcctcccaggtttttcgtctttattctgagcttatgaaagtgaatgttagagttccagctaataccgatttaactcgtattgaaccaattggcatttggatttcaagtgaaccaggacaagggaaatctttttttacgcatatgcttagtacctcccttttaaaggcatgtaacctggagggtatttatactaaccccacaggatctgagtttatggatggttatgtgggtcaggacatccacattattgatgatgctgggcaaaatcgggatgaaaaggacctagccttgttgtgccaatgtatatcgtctgtgccattcacagttcccatggcagatttggctgagaaaggaactttctataccagcaaggtagttatagccacaaccaataagatggattttaactgcatggtgttaactgaccctggtgcattggaacgccgatttccatttaacttgagagtgcgggcagtggctagctttatgacaaaggatagaaagcttgatgtcccacgctccatgggtgcaatggctgatggttcatgctgggaatgttctacagattatggtcgatcctggtcccctgtgaatatgaaggagcttgttaagaaaattgcagatgtttataaagagagagatgatgccttaactgtttggaaatataagttaaatcagattagaaatgaaatggttccgggagattcaattggcaggattttggaacctatggaggagactctttgttctcttgagagacgctttggccaacttgcagatagcctgagggagaactatcataaaacagctgatgagttaattgaggctattgaggatttaatggcacctggccagagccctttttcctgctttgaacccacaggacccactattaaatatacagcctgtgataaggttaaatcttgggttaaaaatcatatgaaaagatggagtgattttgtgattaggaataaaggctggtttacattattttctgtactttcttcttttctttctattcttaccttggtttatcttcattataagaaagagaaaaaagaggataaacaggagcgggcctacaaccctcaaactgcaattcccaagaagggaggtaggccgaagctcacattaatgaagagtactaattttgttaatgaggcaccatatatgcaagatttagaacattgctttgcccaaacagcctatatatcatcaagtgaaacaacagatattatacattgttccgctctttttgaggataccatattggtatatggccattcacagtttttttttaaccgctatgaagatttgcggttacattttaaaggagcaatttttcccattgagggtggccgcatttcccaggttacagtgaatgggcaaccaatggatttaattatggttaaaattgataaattgcccataacttttaaaaattatacaaaatattataccagagatgttggcaaggataatcttttaatttggaattctgataagggtagacttgctatgcctgtagattgtgtggtgccagcaggaccaatagagacaatggaaggtaccataacacataaaacatattcctataaggttgcttccaaacgaggcatgtgtggtggtctcttggtcacaagagtcaatggtacctttaagatcttggggatgcatatagcaggtaatggacagatggcgcgggccgctgctgttcattttattaccaatggcaatatgcaagatcaaggtatagtcgtgaaaaaggaaaagttagaaaaacccatgttcttgccctccaagactgctttgagtcccagcccccttaatggaattgttcctgtgaaaatggaaccagctgttttgagcccccatgatgcccgtttggaagttgtaatgccaagtgtggtgaaaagtgcagctgccaagtatcgtgttaatatttttaatcctgattacgatatttgggagcgggttgttgatgatttgaaagttagatttagacagaaattgggcatacataaacataccaccattcagaaagcagtgcaggggttttcttcattggcttccttagatttggctacctcaccgggcttgaagtatgtttcccaaggacttaagaaacgagatcttttgagtgtcgagcctttttgggttagtccccaattggaaatggatgttaaacaaattcttgctgatgtttattctggtaataaaccaaccacccaatttgcagcacatttgaaagatgaattgaggaagaaagaaaaagtggctcaaggcaaaactcgctgtatcgaggcttgttcagtggattttgtggtggcttatagagttgttatgtcttctctttatgaagccatctaccaaacaccagcacaggagttaggccttgcagtaggcatgaatccatggactgattgggatccaatgattaatacccttcttccctataattatggcctggattattcctcctatgatggtagtctttcagagcagttaatggcatatggtgttgatattttggcctattgccatgaagagcctgaggttgtgaaattgttgcatagccctataataaattcccagcatgttgttatggatgagctatggcatgtgatggggggaatgccctcaggtgctccttgcacaactgttcttaattctatttgtaatcttcttgtttgttcctatttagcctatgaacaaagtttggatttggaggtaagaccaattgtgtatggggatgatgtgatattttctgtttcctcaccattggatatggactattttgtgaagagtgctgcagagacgtttggtatggaagtgacggcctctgataaaaatggtgcccccaaattgcttagaatggaggagatagaatttttaaaaagaactacgaaaatgtttcccggctctacctataaggtaggggccctgagcctggatactatggaacagcatataatgtggatgaaaaatttatccacattcccagaacaattggtcagttttgaaaatgaattggctttgcatgggaaggatgtttatgatgattataaacaaaagtttaatgttgttttgaatcagtggcgagtgtgcatgcaggattatgatgtggtgctgcatcgtatgcttcgctatgtttttgagtgattcttttctttttgctttgctttttgctttgcttttagtttagttttgcttttgcttttaaaagattttgatttgattgaattaggcccaccaatc

SEQ ID NO: 2 - RNA, 737 nt, Ljungan virus
  LV 87-012 strain
uuugaaaggggucuccuggugggguggguacacuucucgcucgaugaguggggguguggcucauugcccacaccugguugguucccagguucauacaauaaccaucaauaaacuuuuaacaucuaagauaguauuaucccauacuagacuggacgaagccgcuuggaauaagucuagucuuaucuuguauguguccugcacugaacuuguuucugucucuggagugcucuacacuucaguaggggcuguacccgggcggucccacucuucacaggaaucugcacagguggcuuucaccucuggacagugcauuccacacccgcuccacgguagaagaugaugugugucuuugcuugugaaaagcuugugaaaaucguguguaggcguagcggcuacuugagugccagcggauuaccccuagugguaacacuagccucugggcccaaaaggcaugucauuugaccacucagguacacaaccccagugaugcacacgcuuaguaauggcuuaguaacaaacauugauugaucauuugaaagcuguuaggagguuuagguaugacgggcugaaggaugcccugaagguacccauagguaaccuuaagcgacuauggaucugaucaggggcccaccauguaacacauggguagaagucuucggaccuuggguuaaaaaacgucuaggcccgccccccacagggaugugggguuucccuuauaaccccaauauuguauaaug

SEQ ID NO: 3 - RNA, 734 nt, Ljungan virus
  145SL strain
uuugaaaggggucuccuggugggguggguacgucucucgcucauugagugggggcguggcucaccaaccacaccugguugguccccagguucaugcaauaaccacuuuuuguaaucuuuacaucuaagcuuaauucacccacuagaacuggacgaaaccgcuuggaauaaguuugguucucucuugaguguguuuuguguuagcauuauuucugucucuagagugcuuuacacucuaguaggggcuguacccgggcggucccacucuucacaggaaucugcacagguggcuuucaccucuggacagugcauuccauacccgcuccacaauagaagaugauguauaucuuuguuugugaaaugcucaugaaacguguguguaggcguagcggcuacuugaaugccagcggaaccccccuagugguaacacuagccucugggcccaaaaggcaugucucugaccauucaaguacacaaccccagugauacacacauuuaguaauggcucaguaauggacauugauugaucaucagacaauuguuaggaggccuagguaugacgggcugaaggaugcccuggagguacccgcagguaaccuuaagagacuguggaucugaccaggggcccaccauggaaacauggguagaagucuucggaccuuggguuaaaaaacgucuaggcccgccccccacagggaugugggguuucccuuauaaccccaauaucacauuaug

SEQ ID NO: 4 - RNA, 738 nt, Ljungan virus
  174F strain
uuugaaaggggucuccuggugggguggguacacuccucgcucaaugaguggggguguggcucauugcccacaccugguugguucccagguucauacaauaaccaucaauaaacuucucaacaucuaagauaguacuaucccacacuaaacuggacgaagccgcuuggaauaagucuaguuucauucuguguguguuuugcacugaaauuauuucugucucuggggugcucuacacuucaguaggggcuguacccgggcggucccacucuucacaggaaucugcacagguggcuuucaccucuggacagugcauuccacacccgcuccacaguagaagaugaugugugucuuugcuugugaaaagcuugugaaaaucguguguaggcguagcggcuacuugagugccagcggacuaccccuagugguaacacuagccucugggcccaaaaggcaugucaauugaccacucagguacacaaccccagugaugcacacgucuaguaacggcuuaguaacaggcauugauugaucauuugaaaacugcuaggagguuuagguaugacgggcugaaggaugcccugaagguacccauagguaaccuuaagcgacuauggaucugaucaggggcccaccauguacuacauggguagaagucuucggaccuuggguuaaaaaacgucuaggcccgccccccacagggaugugggguuucccuuauaaccccaauauuguauaaug

SEQ ID NO: 5 - RNA, 113 nt, Ljungan virus
ugagugauucuuuucuuuuugcuuugcuuuuugcuuugcuuuuaguuuaguuuugcuuuugcuuuuaaaagauuuugauuugauugaauuaggcccaccaaucaaaaaaaaaa

SEQ ID NO: 6 - RNA, 128 nt, Ljungan virus
ugauugaauugauuuaguuugauuuugauuuuauuagcuuuaguuuauguaaguuagaauuagauuauuuuaguuuaguuuuaaagauuuugauuugauugaauuuggcccaccaaucaaaaaaaaaa

(19) United States
(12) Patent Application Publication
(10) Pub. No.: US 2005/0233311 A1
(43) Pub. Date: Oct. 20, 2005

(54) Family of picornaviruses

(76) Inventors: Michael A. Lindberg, Kalmar (SE); Susanne Johansson, New Castle (AU); Bo Niklasson, Stockholm (SE)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/512,392

(22) PCT Filed: Apr. 23, 2003
(86) PCT No.: PCT/IB03/02227

§ 371 Date: Jun. 15, 2005

Related U.S. Application Data
(60) Provisional application No. 60/375,328, filed on Apr. 23, 2002.

(51) Int. Cl. C12Q 1/70; C07H 21/04; C12N 7/00; C07K 14/085; C07K 16/10

(52) U.S. Cl. 435/5; 435/235.1; 530/350; 530/388.3; 536/23.72

(57) ABSTRACT

The present invention relates to a new family of viruses within the Picornavirus family. The present invention also relates to an isolated nucleic acid from said virus or fragments of said nucleic acid. The present invention also relates to an isolated protein of said virus or an immunogenic fragment thereof. The invention also relates to antibodies or antigen-binding fragments of such antibodies directed against said virus. The present invention also relates to diagnostic kits. The present invention also relates to a method of detecting the presence of said virus in a subject suspected of having a viral infection.

### TECHNICAL FIELD OF THE INVENTION

The present invention relates to a new family of viruses within the Picornavirus family. The present invention also relates to an isolated nucleic acid from said virus or fragments of said nucleic acid. The present invention also relates to an isolated protein of said virus or an immunogenic fragment thereof. The invention also relates to antibodies or antigen-binding fragments of such antibodies directed against said virus. The present invention also relates to diagnostic kits. The present invention also relates to a method of detecting the presence of said virus in a subject suspected of having a viral infection.

### BACKGROUND OF THE INVENTION

Ljungan virus, belonging to the Picornavirus family, is carried by small rodents and causes a number of diseases in animals, including humans. The partial sequences of the first three serologically related strains of Ljungan virus isolated from Sweden were initially disclosed in the International Patent Application WO 98/11133, incorporated herein by reference. The full sequences of these three Ljungan virus isolates, 87-012 (Genbank accession number AF327920), 174F (Genbank accession number AF327921) and 145SL (Genbank accession number AF327922), were subsequently published in Johansson S. et al., Journal of Virology, 76(17): 8920-8930 (2000).

Recently, the near complete genomic sequence of another virus, M1146 (Johnson's Microtus montanus enterovirus USA M-1146) isolated in 1962 from voles trapped in Oregon, USA, has led to the classification of another putative member of the Picornavirus family, being most closely related to the three known Swedish Ljungan viruses (Johansson S. et al., Journal of General Virology, 84:837-844 (2003). The strain M1146 possesses typical Ljungan virus genomic organization including a cluster of two 2A homologues. There are significant differences throughout the capsid protein region, while the non-structural region of M1146 is closely related to the Swedish Ljungan virus genomes. Genetic and phylogenetic analyses show that M1146 represents a new genotype within the distinct Ljungan virus cluster.

Ljungan viruses have been shown to infect humans and to be involved in various human diseases. Serological studies have indicated the presence of Ljungan virus in both children and adult patients diagnosed with Diabetes Mellitus as well as patients dying from myocarditis (see WO 98/11133, incorporated herein by reference). Furthermore, sera from children with type 1 diabetes have detectable circulating Ljungan virus antibodies.

Rodents, such as bank voles, are well-known reservoirs and vectors for viruses causing diseases in humans. It has been shown in epidemiological studies that the incidence of type 1 diabetes in humans track the 3 to 4 year population density cycles of bank voles (Clethriionomys glareolus) with a similar time lag (Niklasson, N. et al., Emerg. Infect. Dis., 4:187-193 (1998)) and this has been associated with picornavirus infection. It has also been observed that the incidence of patients dying from myocarditis as well as the number of patients diagnosed with Guillain Barré Syndrome, also track the 3 to 4 year population fluctuations of bank voles with different time delays (see WO 98/11133, incorporated herein by reference). Thus, infection with picornaviruses such as Ljungan virus, may be important in the development and pathology of a number of diseases affecting humans including Myocarditis, Cardiomyopathia, Guillain Barré Syndrome, Diabetes Mellitus, Multiple Sclerosis, Chronic Fatigue Syndrome, Myasthenia Gravis, Amyothrophic Lateral Sclerosis, Dermatomyositis, Polymyositis, Spontaneous Abortion, and Sudden Infant Death, Bell's (facial) paralysis, Addison's disease and Pernicous anemia.

Thus, there is a need for the development of new compounds and methods to treat or prevent infection by such viruses.

### SUMMARY OF THE INVENTION

The invention provides for a new family of viruses within the Picornavirus family. The invention also provides for an isolated nucleic acid from said virus or fragments of said nucleic acid. The invention also provides for an isolated protein of said virus or an immunogenic fragment thereof. The invention also provides for antibodies or antigen-binding fragments of such antibodies directed against said virus. The invention further provides for diagnostic kits. The invention further provides for a method of detecting the presence of said virus in a subject suspected of having a viral infection.

### BRIEF DESCRIPTION OF THE DRAWINGS

FIG. 1 shows the nucleotide sequence of Ljungan virus isolate M1146 (Genbank accession number AF538689).

FIG. 2 shows the predicted stem-loop structures of the 5′UTR region of the virus of the invention.

FIG. 3 shows the predicted secondary structures for the 3′UTR region of the virus of the invention.

FIG. 4 shows a schematic representation of the genomic organization of the virus of the invention. The polyprotein encoding region (boxed) is flanked by the 5′untranslated region (UTR) and the 3′UTR which contains a poly(A) tail at the end. The protein products within the polyprotein region are indicated. An enlargement of the VP1-2A region of the viral genome is also shown.

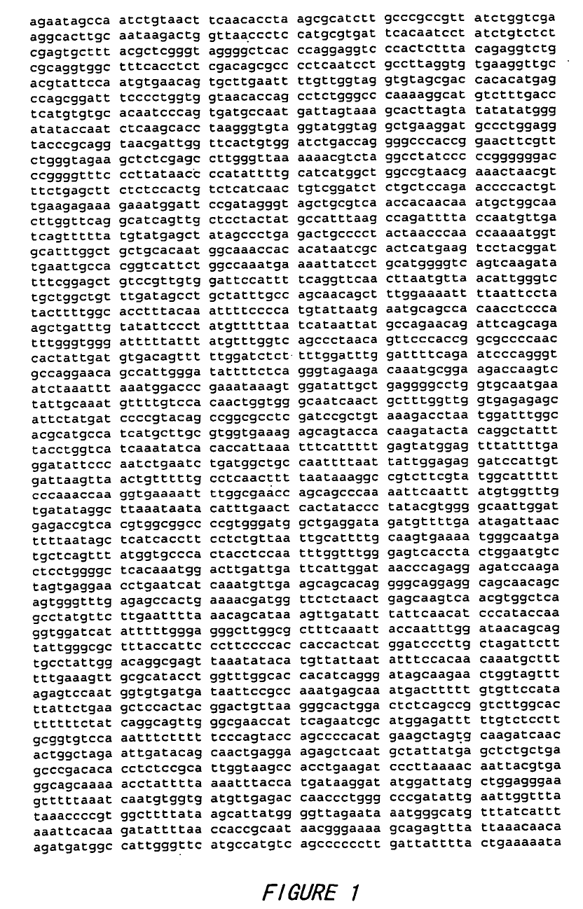

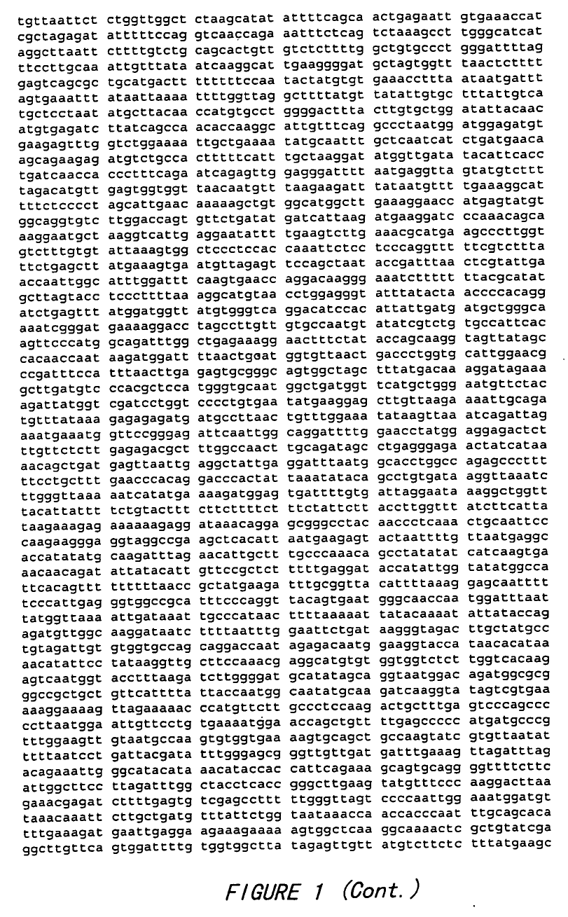

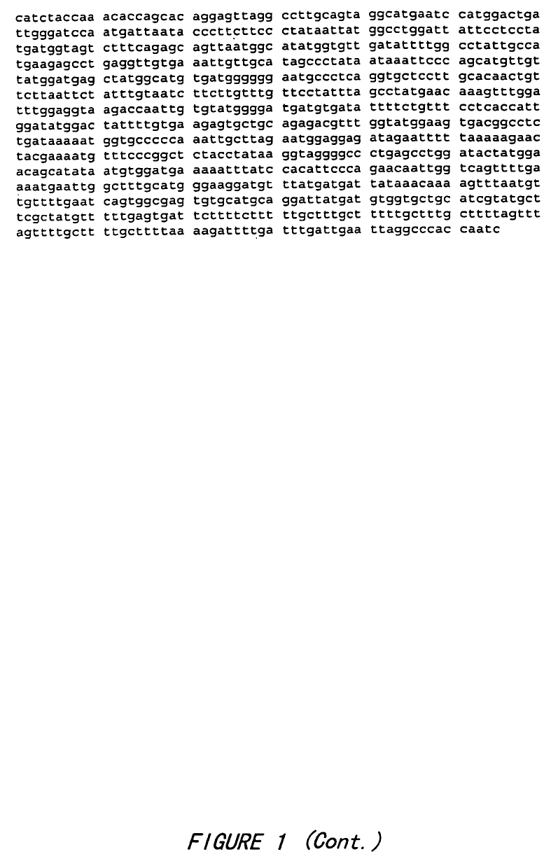

### DETAILED DESCRIPTION OF THE INVENTION

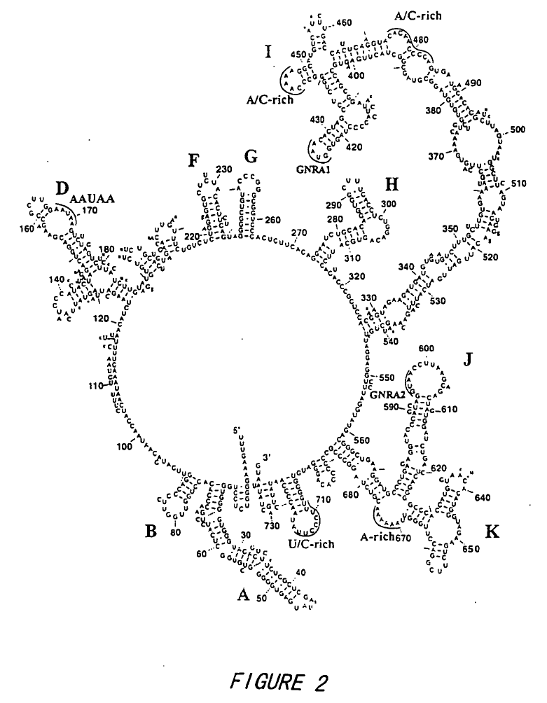

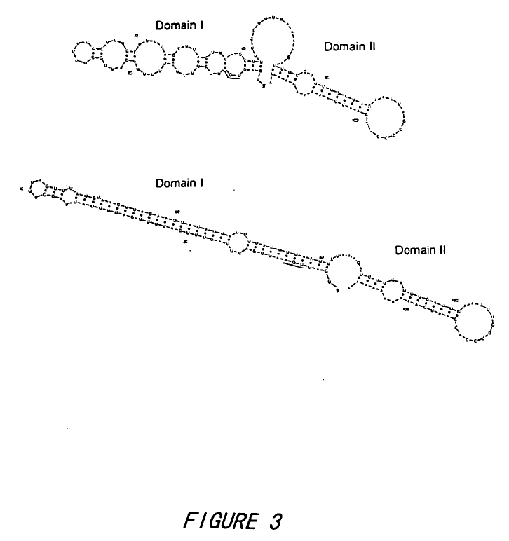

According to the first embodiment of the present invention, there is provided a novel family of viruses within the Picornavirus family. The virus family is characterized by various structural and non-structural features common to the members of the family. Such identifying features of the viruses include a 5′ untranslated region (5′UTR) stem-loop structure (FIG. 2), a unique 3′UTR region (FIG. 3), non-structural proteins that are larger than in other picornaviruses, the presence of a cluster of two unrelated 2A proteins, an amino acid extension of the VP1 protein and the expression of a VP1-2A1 “fusion” protein.

The UTRs of picornaviruses are characterized by complex secondary structures, which serve for initiation of translation and RNA replication (see Racaniello, V. R., (2001). Picornaviridae: The viruses and their replication. In Fields virology 4th ed., 4th edn, pp. 685-722. Edited by D. M. Knipe, P. M. Howley, D. E. Griffin, R. A. Lamb, M. A. Matrin, B. Riozman & S. E. Straus. Philadelphia: Lippincott Williams & Wilkins). Initiation of translation is dependent on the tertiary structure in the 5′UTR referred to as the internal ribosome entry site (IRES).

In the viruses of the invention, the 5′ UTR precedes the putative initiation codons, which are located in an optimal Kozak context. It has been previously observed that this region includes the type II IRES in all picornaviruses except entero-, rhino-, and hepatoviruses. In the viruses of the invention, counterparts to the three major 3′-located secondary-structure elements, I, J, and K, of the type II IRES all formed in the 5′ UTR (FIG. 2). The apical parts of the I domain, including the prominent GNRA tetranucleotide (GNRA1) and one of two A/C-rich loop regions, and J domain, as well as the A-rich loop at the junction of the J and K domains, are conserved at the sequence level (FIG. 2). Additionally, a oligopyrimidine tract (U/C-rich region) occurs a short distance upstream of the start codon in an atypically well-structured region. The 5′ half of the 5′ UTR of the viruses of the invention may also contain stem-loop structures found in other group II picornaviruses, although no equivalent to element C may be present.

In some embodiments, the viruses of the invention are characterized by a 3′UTR, the 3′ third of which is highly conserved both in primary sequence and in the predicted stem-loop structure thereof (domain II) (FIG. 3)

The viruses of the invention possess a full complement of nonstructural proteins identified previously in parechoviruses; however, they are larger in the instant viruses than in parechoviruses, mostly because of extra sequences at one or both termini.

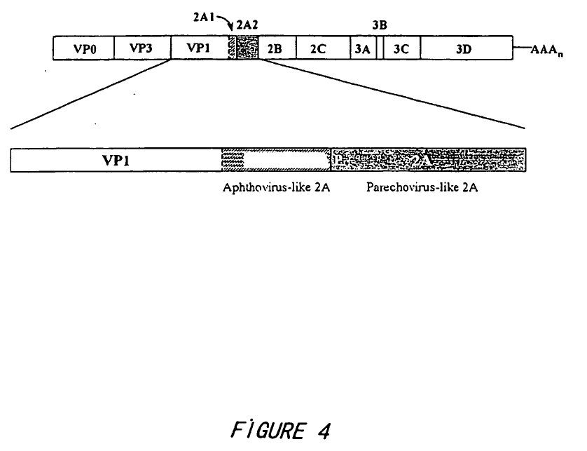

A distinctive feature of the viruses of the invention is the presence of two 2A proteins (2A1 and 2A2) rather than one. The N-terminal 2A1 is related to the NPGP family of 2As encoded by cardio-, erbo-, tescho-, and aphthoviruses. The C-terminal 2A2 belongs to the H-NC family of 2As encoded by parechoviruses, kobuviruses, and avian encephalomyelitis virus. The unprecendented association of two structurally different 2As may result in a unique regulation of the life cycle of the viruses. In some embodiments, the 2A1 protein may mediate the separation between 2A1 and 2A2. In some embodiments, the 2A1 protein is expressed as a VP1-2A1 “fusion” protein (FIG. 4).

Another feature of the viruses of the invention is the sequence at the C terminus of VP1. Specifically, this region of the virus contains a distinct amino acid extension. Typically, the extension is about 40-50 amino acids. In some embodiments, the VP1 extension is 41, 42, 43, 44, 45, 46, 47, 48 or 49 amino acids.

A virus of the invention includes a complete virus or any component of the virus such as the viral capsid and viral subunits. The invention also includes a virus in attenuated or killed form.

The viruses of the invention are useful, e.g., in methods and compositions to treat, prevent or lessen the severity of infection by the virus or conditions or diseases caused by such infection. The viruses also are useful for the production of antibodies for use as diagnostic reagents or in therapeutic uses as described. Detection of the virus in a subject is useful to identify the risk of developing a condition or disease caused by infection with the virus. The virus also is useful to screen for and identify compounds for therapeutic use.

Another aspect of the invention is an isolated nucleic acid from a virus of the invention or fragments of said nucleic acid. The nucleic acid may be RNA or DNA, including cDNA. An isolated nucleic acid of the invention may encode a structural protein of a virus of the invention or an immunogenic fragment of said structural protein. In some embodiments, the isolated nucleic acid encodes a non-structural protein of the virus of the invention or an immunogenic fragment thereof. In other embodiments, the isolated nucleic acid is all or part of the 5′UTR or the 3′UTR. The isolated nucleic acid of the invention are useful, e.g., as probes for detecting the presence of a virus of the invention, as a primer to amplify all or a portion of the viral genome or for the production of viral structural or non-structural proteins.

Another aspect of the invention is an isolated protein of a virus of the invention or an immunogenic fragment thereof. The isolated proteins and fragments thereof are useful in therapeutic or prophylactic compositions, as diagnostic reagents and to generate antibodies that specifically bind to the structural or non-structural proteins of the virus. The viral proteins and fragments thereof also are useful as vaccines to prevent or inhibit infection with a virus of the invention or a disease or condition caused by such infection. In some embodiment, the disease or condition caused by the virus of the invention is selected from the group consisting of Myocarditis, Cardiomyopathia, Guillain Barré Syndrome, Diabetes Mellitus, Multiple Sclerosis, Chronic Fatigue Syndrome, Myasthenia Gravis, Amyothrophic Lateral Sclerosis, Dermatomyositis, Polymyositis, Spontaneous Abortion, Sudden Infant Death Syndrome, Bell's (facial) paralysis, Addison's disease, and Pernicious anemia.

A further aspect of the invention is antibodies directed against a virus of the invention or antigen-binding fragments of such antibodies. The antibodies can be polyclonal or monoclonal. The antibodies of the invention can be human, non-human or humanized antibodies.

A further aspect of the invention is diagnostic kits comprising a component selected from a virus of the invention, an isolated nucleic acid from a virus of the invention or fragments of said nucleic acid, an isolated protein of a virus of the invention or an immunogenic fragment thereof and antibodies or antigen-binding fragments of such antibodies directed against a virus of the invention.

1. A picornavirus characterized by a virus genome comprising two 2A genes, excluding the Ljungan virus isolates 87-012, 174F, 145SL and virus isolate M1146.

2. The virus of claim 1, excluding viruses comprising in their virus genome a 5′UTR that is at least 75% homologous to the 5′UTR of Ljungan virus 87-012 (Genbank accession number AF327920).

3. The virus according to claim 1 or 2, wherein the virus genome is further characterized by one or more of the following features:
(a) a 5′UTR stem-loop structure substantially as shown in FIG. 2,
(b) a 3′UTR domain II stem-loop structure substantially as shown in FIG. 3,
(c) an amino acid extension of the VP1 protein of about 40 to about 50 amino acids compared to the VP1 of parechoviruses, and
(d) expresses a VP1-2A1 fusion protein.

4. An isolated nucleic acid or a fragment thereof from a virus according to any one of claims 1-3.

5. An isolated polypeptide or an immunogenic fragment thereof of a virus according to any one of claims 1-3.

6. An isolated antibody or an antigen-binding fragment thereof that specifically binds to a virus according to any one of claims 1-3 or a polypeptide or an immunogenic fragment thereof according to claim 5.

7. The virus according to any one of claims 1-3, wherein the virus genome encodes a complete virus or any component of the virus such as the viral capsid and viral subunits.

8. The virus according to any one of claims 1-3, wherein the virus is in attenuated form or killed form.

9. A diagnostic kit comprising at least one one member selected from the group consisting of:
(a) the virus according to any one of claims 1-3,
(b) the nucleic acid or a fragment of thereof according to claim 4,
(c) the polypeptide or an immunogenic fragment thereof according to claim 5, and
(d) the antibody or antigen-binding fragment thereof according to claim 6.

10. A method of detecting the presence of a virus according to any one of claims 1-3, comprising contacting a sample from a subject suspected of having a viral infection with a reagent selected from the group consisting of:
(a) the virus according to any one of claims 1-3,
(b) the nucleic acid or a fragment of thereof according to claim 4,
(c) the polypeptide or an immunogenic fragment thereof according to claim 5, and
(d) the antibody or antigen-binding fragment thereof according to claim 6.
